(19) United States
(12) Patent Application Publication
(10) Pub. No.: US 2005/0003356 A1
(43) Pub. Date: Jan. 6, 2005

(54) Diagnostic methods for cardiovascular disease, low hdl-cholesterol levels, and high triglyceride levels

(76) Inventors: Michael R. Hayden, Vancouver (CA); Karin Yvon Zwarts, Ms Amsterdam (NL); Susanne M. Clee, Madison, WI (US)

Correspondence Address: BOZICEVIC, FIELD & FRANCIS LLP, 1900 UNIVERSITY AVE, SUITE 200, EAST PALO ALTO, CA, 94303, US

(21) Appl. No.: 10/479,198

(22) PCT Filed: May 24, 2002
(86) PCT No.: PCT/CA02/00761

Related U.S. Application Data
(60) Provisional application No. 60/293,742, filed on May 25, 2001.

(51) Int. Cl. C12Q 1/68

(52) U.S. Cl. 435/6

(57) ABSTRACT

A method for determining propensity toward developing a cardiovascular disease in a patient at risk thereof by determining the presence in an ABCA1 gene of said patient of a polymorphism in the DNA sequence of the gene wherein said polymorphism is present in a non-coding region of said gene is disclosed. Also described is a method of identifying a modulator of ABCA1 polynucleotide expression comprising by determining the ability of a test compound to modulate the activity of a polynucleotide comprising a polymorphism disclosed herein, or to mimic the effects of such polymorphism where such effects are beneficial. Treatment of cardiovascular disease, especially coronary artery disease, using agents identified by the disclosed methods is also described.

This application claims priority of U.S. Provisional Application 60/293,742, filed 25 May 2001, the disclosure of which is hereby incorporated by reference in its entirety.

### FIELD OF THE INVENTION

The present invention relates to the field of gene polymorphisms, especially single nucleotide polymorphisms present in non-coding regions of the ABCA1 gene, and their use in diagnosing risk of cardiovascular disease, including coronary artery disease, and in screening for compounds useful in mimicking advantageous polymorphisms and for agents that enhance the activity of beneficial polymorphisms.

### BACKGROUND OF THE INVENTION

Atherosclerotic cardiovascular disease is the leading cause of death worldwide¹. Altered lipoprotein levels are pivotal risk factors for atherosclerosis^{2,3}. In particular, low HDL cholesterol (HDL-C) levels are a major independent risk factor for the development of premature coronary disease⁴⁻⁶. The anti-atherogenic function of HDL is generally attributed to its role in reverse cholesterol transport (RCT), whereby excess cholesterol is transported from peripheral cells to HDL particles for subsequent delivery to the liver^{7,8}. The protein crucial for the initial step of RCT, namely ABC1, was recently identified⁹⁻¹².

Complete ABC1 deficiency is the underlying cause of Tangier disease (TD)^{9,11,12}, a rare disorder associated with a near absence of HDL-C and apolipoprotein AI and with remarkably decreased cholesterol efflux from cells¹³. Clinically, TD is associated with hepatosplenomegaly, neuropathy and cholesterol ester accumulation in specific cells¹³. Individuals heterozygous for ABC1 mutations are characterized by low HDL-C levels, increased triglycerides (TG), depressed levels of cholesterol efflux and an increased risk of coronary artery disease (CAD), but have no obvious clinical manifestations of cholesterol ester accumulation^{9,10,14}. Cholesterol efflux levels are highly correlated with HDL-C levels in these individuals¹⁴. The frequency of individuals with severe mutations in the ABC1 gene is low, but common variants having minor functional effects could be of great clinical relevance for the general population.

We have previously shown that individuals heterozygous for mutations in the ABC1 gene (also called ABCA1) have decreased HDL cholesterol (HDL-C), increased triglycerides (TG) and a greater than threefold increased frequency of coronary artery disease (CAD) and that single nucleotide polymorphisms in the coding region (cSNPs) of the ABC1 gene may significantly impact plasma lipid levels and the severity of CAD in the general population. We have now identified several SNPs in non-coding regions of ABC1 that may be important for the appropriate regulation of ABC1 expression (i.e. in the promoter, intron 1 and the 5′ untranslated region (UTR)), and have examined the phenotypic effects of these SNPs in the REGRESS population. Of 12 SNPs, 4 were associated with a clinical outcome. A 3-fold increase in coronary events and an increased family history of CAD was evident for the G-191C variant. Similarly, the C69T SNP was also associated with a 2-fold increase in events. In contrast, the C-17G was associated with decreased coronary events, and the InsG319 SNP was associated with less focal and diffuse atherosclerosis. For all these SNPs, the changes in atherosclerosis and CAD occurred independent of changes in plasma lipid levels, findings which were replicated in a second cohort. These data suggest that common variation in non-coding regions of ABC1 may significantly alter the severity of atherosclerosis, without necessarily influencing plasma lipid levels.

We have previously presented a complete analysis of 10 single nucleotide polymorphisms in the coding region of the ABC1 gene (cSNPs)¹⁵. We have shown that cSNPs of the ABC1 gene influence plasma lipid levels and the severity of CAD. Interestingly, the R219K cSNP is associated with decreased TG, increased HDL-C and a decreased severity of CAD, compatible with a gain of function, while other cSNPs were associated with more moderate effects¹⁵.

Here, we describe 12 non-coding SNPs in potential regulatory regions and have examined the functional effects of these SNPs in the promoter, the 5′ untranslated region (UTR) and first intron. Several studies have shown that SNPs in these regions from other genes indeed have functional consequences ¹⁶⁻¹⁸. We have also recently shown that sequences within the first intron of ABC1 constitute an alternate promoter with three alternate transcription start sites, and thus may have direct effects on the regulation of ABC1 (Singaraja et al, manuscript submitted). An alternate transcription start site within intron 1 has also recently been reported by another group¹⁹.

We have now examined the phenotypic effects of these 12 non-coding SNPs in a large ethnically uniform cohort (REGRESS) and show that they indeed are associated with altered risk and severity of CAD, without associated changes in lipid and lipoprotein levels. This provides evidence that sequences in these regions are important for the proper regulation of ABC1 and suggest that changes in ABC1 regulation can alter risk for CAD presumably through influencing RCT without necessarily having an effect on lipid levels.

### BRIEF SUMMARY OF THE INVENTION

In one aspect, the present invention relates to a method for determining propensity toward developing a cardiovascular disease in a patient at risk of developing said disease comprising determining the presence in an ABCA1 gene of said patient of a polymorphism in the DNA sequence of said gene wherein said polymorphism is present in a non-coding region of said gene.

In preferred embodiments, the polymorphism is present in the promoter region of said gene or in an intronic region.

In preferred embodiments, the disease is coronary artery disease or atherosclerosis, or a disease that involves increased triglyceride or cholesterol levels, or decreased HDL-C levels, in a patient, especially in the plasma of said patient.

In preferred embodiments, the disease involves decreased lipid transport in the cells of the patient, especially decreased HDL-C transport.

In additional preferred embodiments, the polymorphism is a single nucleotide polymorphism, most preferably any of the polymorphisms depicted in Table 1 (SEQ ID NOS: 1-24).

In another aspect, the present invention relates to method for identifying a modulator of ABCA1 polynucleotide expression comprising:
(a) contacting a compound with a polynucleotide that encodes ABCA1 polypeptide, which polynucleotide comprises a polymorphism in a non-coding region of said polynucleotide, under conditions promoting said contacting and promoting expression of ABCA1 polypeptide by said polynucleotide;
(b) determining the activity of said polynucleotide in expressing said ABCA1 polypeptide after said contacting wherein a difference in the expression of said polynucleotide relative to when said compound and said polynucleotide are not contacted indicates polynucleotide modulating activity,
thereby identifying a modulator of ABCA1 polynucleotide expression.

In a preferred embodiment, the ABCA1 polynucleotide is present in a cell, which cell then expresses the ABCA1 polypeptide and such expression is readily measured, such as by measuring lipid transport across the membrane of the cell whereby an increase in transport shows increased expression of the polypeptide. Thus, in a preferred embodiment, the difference in expression in step (b) is an increase in expression. Preferably, the polymorphism is present in an intronic region or promoter region, or some other non-coding region, such as an enhancer region, of the polynucleotide.

In a preferred embodiment, the polymorphism is a single nucleotide polymorphism (SNP), most preferably one of the SNPs shown in Table 1 (SEQ ID NOS: 1-24).

Such polymorphisms may also have the effect of decreasing the activity of said polynucleotide.

In a further aspect, the present invention relates to a method for identifying an agent that modulates plasma lipid levels comprising administering to an animal an effective amount of a compound first identified as an ABCA1 modulator using a screening method as disclosed herein. In preferred embodiments thereof, the compound has the effect of reducing plasma triglyceride levels, reducing plasma cholesterol levels, or increasing plasma HDL-C levels.

In an additional aspect, the present invention relates to a method of treating a patient for cardiovascular disease comprising administering to a patient afflicted therewith of an effective amount of a compound first identified as an ABCA1 modulator using a screening method as disclosed herein. In preferred embodiments, the disease is coronary artery disease or atherosclerosis.

In yet a further aspect, the present invention relates to a method of protecting a patient against developing cardiovascular disease comprising administering to a patient at risk thereof of an effective amount of a compound first identified as an ABCA1 modulator using the method as disclosed herein. In preferred embodiments thereof, the disease is coronary artery disease or atherosclerosis.

### BRIEF DESCRIPTION OF THE DRAWINGS

FIG. 1. Schematic diagram of the location of non-coding SNPs in the promoter, intron 1 and the 5′ untranslated regions of exons 1 and 2 of the ABC1 gene. The alternate exons we have recently identified within intron 1 are also indicated (exons 1 b, c, d; Singaraja et al, manuscript submitted). The translation initiation (ATG) site in exon 2 is indicated. SNPs that are in complete or near complete linkage disequilibrium are joined by dashed lines. Variants marked by a * were previously reported in reference²¹. The diagram is not drawn to scale.

FIG. 2. Event-free survival by G-191C genotype. The curves represent the cumulative proportion of the cohort that was event-free during the trial (thin line for AA, thick line for AB and dashed line for BB). Homozygous carriers of this variant (BB) had significantly more events during the two-year trial than individuals with either of the other genotypes.

FIG. 3. Event-free survival in C69T carriers and non-carriers. The curves (AB+BB− thick line, AA− thin line) represent the cumulative proportion of the cohort that was event-free during the trial. Carriers of this variant had significantly more events during the two-year trial than non-carriers.

### DETAILED DESCRIPTION OF THE INVENTION

The present invention features diagnostically relevant polymorphisms of the human ABC1 gene regulatory region. In particular, we have determined the statistical relationship between certain SNPs in the regulatory domain of the ABC1 gene and the incidence of coronary events and coronary artery disease in humans. This relationship establishes the importance and utility of diagnostic assays which identify the presence or absence of such SNPs in a human. For example, identification of these SNPs can have medical use in (1) diagnosis of disease and predicting disease progression; (2) selection of drugs for patients based on improved efficacy or reduced side-effects; and (3) selection of patients for enrolment in clinical trials and classification of patients in clinical trials by ABC1 genotype. These polymorphisms are also useful in any of the diagnostic assays involving ABC1 nucleic acids or proteins that are described in PCT Publication WO 00/55318, filed Mar. 15, 2000; U.S. Utility application Ser. No. 09/526,193, filed Mar. 15, 2000; PCT Publication WO 00/0115676, filed Sep. 1, 2000; or U.S. Utility application Ser. No. 09/654,323, filed Sep. 1, 2000 (which are each herein incorporated by reference). In addition, see Zwarts et al, ABCA1 regulatory variants influence coronary artery disease independent of effects on plasma lipid levels, Clin. Genet. 61(2): 115-25 (February 2002), the disclosure of which is hereby incorporated by reference in its entirety.

For example, determination of the genetic subtyping of ABC1 gene sequences can be used to subtype individuals or families with lower than normal HDL cholesterol levels or higher than normal triglyceride levels to determine whether the lower than normal HDL or higher than normal triglyceride phenotype is related to ABC1 function. This diagnostic process can lead to the tailoring of drug treatments according to patient genotype (referred to as pharmacogenomics), including prediction of the patient's response (e.g., increased or decreased efficacy or undesired side effects upon administration of a compound or drug). These diagnostic methods may also be used to determine a subject's risk for a cardiovascular disease, such as coronary artery disease, atherosclerosis, myocardial infarction, ischemic attack, angina, peripheral vascular disease, or stroke.

In one such aspect, the invention features a method for predicting a person's response to a drug by determining whether the person has a polymorphism in an ABC1 gene, promoter, or regulatory sequence that alters the person's response to the drug. Examples of therapeutic agents that can be used in these methods include triglyceride-lowering drugs, HDL cholesterol-raising drugs, and agents for the treatment or prevention of cardiovascular disease, such as coronary artery disease.

In another aspect, the invention features a method of determining a subject's propensity for a disease or condition selected from the group consisting of a lower than normal HDL cholesterol level, a higher than normal triglyceride level, and a cardiovascular disease. This method involves determining the presence or absence of at least one ABC1 polymorphism in the polynucleotide sequence of an ABC1 regulatory region, promoter, or coding sequence or in the amino acid sequence of an ABC1 protein in a sample obtained from the subject, wherein the presence or absence of the ABC1 polymorphism is indicative of a risk for the disease or condition. Desirably, the method also includes analyzing at least five ABC1 polymorphic sites in the polynucleotide sequence or the amino acid sequence.

In yet another aspect, the invention features a method for determining whether an ABC1 polymorphism is indicative of a risk in a subject for a disease or condition selected from the group consisting of a lower than normal HDL cholesterol level, a higher than normal triglyceride level, and a cardiovascular disease. The method includes (a) determining whether the prevalence of the disease or condition in a first subject or set of subjects differs from the prevalence of the disease or condition in a second subject or set of subjects; (b) analyzing the polynucleotide sequence of an ABC1 regulatory region, promoter, or coding sequence or the amino acid sequence of an ABC1 protein in a sample obtained from the first subject or set of subjects and the second subject or set of subjects; and (c) determining whether at least one ABC1 polymorphism differs between the first subject or set of subjects and the second subject or set of subjects, wherein the presence or absence of the ABC1 polymorphism is correlated with the prevalence of the disease or condition, thereby determining whether the ABC1 polymorphism is indicative of the risk. Desirably, the method further includes analyzing at least five ABC1 polymorphic sites in the polynucleotide sequence of an ABC1 regulatory region, promoter, or coding sequence or in the amino acid sequence of ABC1.

In another aspect, the invention provides an electronic database having a plurality of sequence records of ABC1 polymorphisms correlated to records of predisposition to or prevalence of a disease or condition selected from the group consisting of a lower than normal HDL cholesterol level, a higher than normal triglyceride level, and a cardiovascular disease.

In another aspect, the invention features a method for selecting a desirable therapy for modulating ABC1 activity or expression in a subject. This method includes (a) determining the presence or absence of at least one ABC1 polymorphism in the polynucleotide sequence of an ABC1 regulatory region, promoter, or coding sequence or in the amino acid sequence of an ABC1 protein in a sample obtained from the subject, wherein the presence or absence of the ABC1 polymorphism is indicative of the safety or efficacy of at least one therapy for modulating ABC1 expression or activity; and (b) determining a desirable therapy for modulating ABC1 expression or activity in the subject. Desirably, the method further includes analyzing at least five ABC1 polymorphic sites in the polynucleotide sequence of an ABC1 regulatory region, promoter, or coding sequence or the amino acids sequence of ABC1.

The invention also includes methods, compositions, and kits which are useful for identification of the herein disclosed SNPs in a subject (e.g., a human).

In embodiments of any of the various aspects of the invention, the polymorphism is one or more of the polymorphisms listed in Table 1 or described herein (SEQ ID NOS: 1-24). In other desirable embodiments, the polymorphism is in the 5′ regulatory region of ABC1.

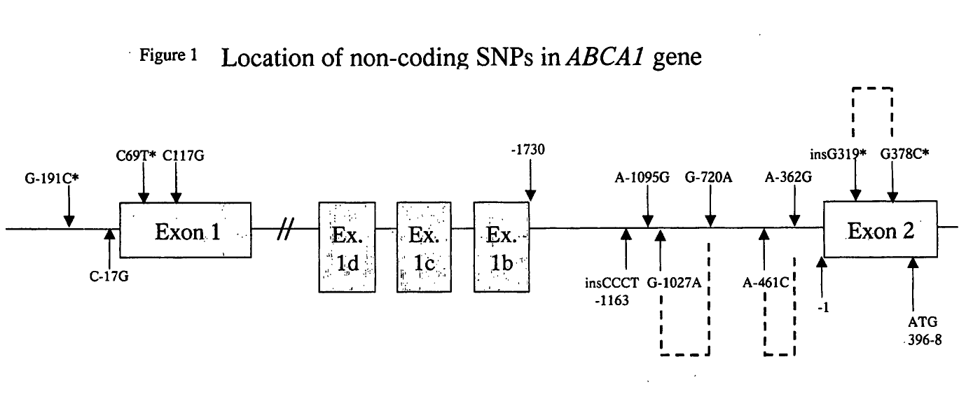

In accordance with the foregoing, the present invention relates to a method for determining propensity toward developing a cardiovascular disease in a patient at risk of developing said disease comprising determining the presence in an ABCA1 gene of said patient of a polymorphism in the DNA sequence of said gene wherein said polymorphism is present in a non-coding region of said gene. As used therein, the polymorphism is present in the promoter region of said gene or in an intronic region or some other non-coding region, as described in FIG. 1, especially where the polymorphism is a single nucleotide polymorphism.

The diseases to be diagnosed include any type of cardiovascular disease, such as, but in no way limited to, coronary artery disease or atherosclerosis, wherein the disease involves increased triglyceride or cholesterol levels, or decreased HDL-C levels, in a patient, especially wherein the plasma levels of the patient reflect these increased or decreased lipid levels. Such diseases also involve decreased lipid transport in the cells of the patient, especially decreased HDL-C transport.

The present invention also contemplates a method for identifying a modulator of ABCA1 polynucleotide expression comprising:
(a) contacting a compound with a polynucleotide that encodes ABCA1 polypeptide, which polynucleotide comprises a polymorphism in a non-coding region of said polynucleotide, under conditions promoting said contacting and promoting expression of ABCA1 polypeptide by said polynucleotide;
(b) determining the activity of said polynucleotide in expressing said ABCA1 polypeptide after said contacting wherein a difference in the expression of said polynucleotide relative to when said compound and said polynucleotide are not contacted indicates polynucleotide modulating activity,
thereby identifying a modulator of ABCA1 polynucleotide expression.

As used in such methods, the ABCA1 polynucleotide may be present in a cell, which cell then expresses the ABCA1 polypeptide and such expression is readily measured, such as by measuring lipid transport across the membrane of the cell whereby an increase in transport shows increased expression of the polypeptide. Thus, in a preferred embodiment, the difference in expression in step (b) is an increase in expression. Preferably, the polymorphism is present in an intronic region or promoter region, or some other non-coding region, such as an enhancer region, of the polynucleotide, especially where the polymorphism is a single nucleotide polymorphism (SNP), most preferably one of the SNPs shown in Table 1 (SEQ ID NOS: 1-24). Such polymorphisms may also have the effect of decreasing the activity of said polynucleotide.

In accordance with the methods of the foregoing, the present invention provides a method for identifying an agent that modulates plasma lipid levels comprising administering to an animal an effective amount of a compound first identified as an ABCA1 modulator using a screening method as disclosed herein. In preferred embodiments thereof, the compound has the effect of reducing plasma triglyceride levels, reducing plasma cholesterol levels, or increasing plasma HDL-C levels.

Because such agents are useful in treating diseases of lipid metabolism, the present invention provides a method of treating a patient for cardiovascular disease comprising administering to a patient afflicted therewith of an effective amount of a compound first identified as an ABCA1 modulator using a screening method as disclosed herein. In preferred embodiments, the disease is coronary artery disease or atherosclerosis.

Agents identified according to the screening assays disclosed herein also find use in preventing lipid-related diseases from developing and thus the present invention provides methods of protecting a patient against developing cardiovascular disease comprising administering to a patient at risk thereof of an effective amount of a compound first identified as an ABCA1 modulator using the method as disclosed herein. In preferred embodiments thereof, the disease is coronary artery disease or atherosclerosis.

The following examples and methodology were used in effecting the disclosure herein.

Identification of SNPs

SNPs in the ABC1 gene were identified during the sequencing of 16 unrelated probands with low HDL-C^{9,10,14} and of BAC (bacterial artificial chromosome) clones spanning the entire region. By definition, SNPs result from the substitution of one nucleotide with another, while other polymorphisms can result from the insertion or deletion of one or more nucleotides²⁰. For simplification, we have used the term SNP to refer to all variants that have been found in our study populations. The UTR SNPs are numbered from the nucleotide described as position 1²¹, naming the first exon number 1. Nucleotides within the promoter are numbered according to their position upstream of the transcription start site, with at −1 as the first nucleotide upstream of that site. The intronic sites are numbered as their position upstream of the 3′ end of intron 1, with the most 3′ nucleotide of the intron as position −1.

Subjects

To assess the effects of these SNPs on lipid levels and CAD, we studied a cohort of 804 Dutch men with proven CAD who participated in the Regression Growth Evaluation Statin Study (REGRESS), which has previously been described²². The REGRESS and its DNA substudies were approved by the institutional review boards and medical ethics committees of all participating centres.

For replication studies, the SNPs were screened in a cohort of individuals with familial hypercholesterolemia, which was available in the lab and has previously been described¹⁵ (and Clee et al, manuscript submitted).

Coronary Artery Disease Measurements

Computer-assisted quantitative coronary angiography was carried out at the start and at the end of the study as previously described²². The mean segment diameter (MSD) measures the average unobstructed diameter along the vessel, a measure of diffuse atherosclerosis. The minimum obstruction diameter (MOD) represents the unobstructed diameter at the site of maximal obstruction, reflecting focal atherosclerosis. Larger measurements of MSD and MOD thus reflect less occlusion of the vessel. The changes in these parameters (delta-MSD and delta-MOD) during the two year study, were calculated as the baseline measurement minus the follow-up measurement. Thus larger values of the delta-MSD and delta-MOD reflect increased progression of coronary atherosclerosis. In addition, the incidence of cardiovascular events (death, myocardial infarction, unscheduled coronary angioplasty or bypass surgery (PTCA, CABG), or stroke/transient ischemic attack) during the study was examined.

In the replication cohort, vascular disease was described as any form of coronary artery disease (myocardial infarction, CABG, PTCA, angina treated with medication, angiographic evidence of CAD), cerebrovascular disease (stroke, transient ischemic attack) or peripheral vascular disease (individuals with claudication and surgery on carotid or abdominal arteries, not including individuals with bruits only, aneurysms, or evidence only from ultrasound).

SNP Screening

For each variant, we identified a restriction enzyme whose cleavage pattern was altered by the variant for development of an RFLP assay. If no suitable enzyme was found, we designed a mismatch primer, whereby a single nucleotide mismatch was incorporated into the primer, creating a restriction site in combination with either the wildtype or variant allele. The specific conditions of all assays are described in Table 1 (SEQ ID NOS: 1-24). All PCR reactions were carried out in the presence of 1.5 μM MgCl₂ (Life Technologies). Thermocycling parameters were as follows: 96° C. for 5 minutes; 33 cycles of 96° C. 10 seconds, 30 seconds at the annealing temperature specified in Table 1, 1 minute at 72° C.; and ended with a final elongation at 72° C. for 10 minutes. All digestions were carried out for 2 hours under the conditions specified by the manufacturer (New England Biolabs).

Large-scale screening of the variants in intron 1 was performed with TaqMan® based PCR assays^{23,24}. Briefly, two fluorogenic hybridization probes (one for each allele) are labelled with different fluorescent reporter dyes at their 5′ terminus and a common quencher dye at their 3′ terminus. The probes are cleaved by the 5′ nuclease activity of Taq enzyme during PCR amplification, separating the reporter dye from the quencher. The fluorescence of each dye in each reaction was normalized to the signal from no-DNA controls and compared to known genotype standards included on each plate.

As a standardised nomenclature for all variants, the allele that was more frequent in the REGRESS population was designated A, while the variant (less frequent) allele was designated B (Table 1 (SEQ ID NOS: 1-24)).

Statistics

We compared the baseline characteristics of the individuals in the REGRESS population in the three genotypes (AA, AB, BB) using one-way analysis of variance, and the chi-square test, where appropriate. We also compared AA versus the combined carrier group (AB+BB) or the homozygous carriers (BB) using a t-test. P-values unadjusted for multiple comparisons are presented to allow the reader to judge the relative significance of the findings. The cumulative event incidence was compared using the logrank test and are presented as Kaplan Meier curves. The change in MOD and MSD and events during the trial were measured following randomization to placebo and pravastatin, which was assessed by chi-square analysis and was equal for all variants except the InsG319, where all 6 BB individuals were randomized to placebo. These parameters were also analyzed in the placebo and pravastatin subgroups separately, and unless otherwise stated, the results for the two treatment groups were comparable, and the combined results are presented. All lipid levels are reported in mmol/L. All values are reported as mean±standard deviation.

Methods for Identifying SNPs in a Patient Sample.

All means of identifying DNA sequences specific to an individual are contemplated by this invention.

In general, the detection of single nucleotide polymorphism and single base mutation or variation requires a discrimination technique, optionally an amplification reaction and optionally a signal generation system. There are numerous techniques available for typing SNPs and allelic variations (for review, see Eberle & Kruglyak Genet Epidemiol 2000;19 Suppl 1:S29-35; Kennedy EXS 2000;89:1-10; Kao et al. Ann Acad Med Singapore May 2000;29(3):376-82; Kao et al. Ann Acad Med Singapore May 2000;29(3):376-82; Landegren et al., Genome Research, Vol. 8, pp. 769-776,1998; Nollau et al, Clin. Chem. 43,1114-1120, 1997 and in standard textbooks, for example ‘Laboratory Protocols for Mutation Detection’, Ed, Landegren, Oxford University Press, 1996 and ‘PCR’ 2nd Edition by Newton and Graham, BIOS Scientific Publishers limited, 1997).

Techniques include direct sequencing (Carothers et al., BioTechniques, Vol. 7, pp. 494-499,1989), single-strand conformation polymorphism (SSCP, Orita et al., Proc. Natl. Acad. Sci. USA, Vol. 86, pp. 2766-2770,1989), allele-specific amplification (Newton et al., Nucleic Acids Research, Vol. 17, pp. 2503-2516,1989), restriction digestion (Day and Humphries, Analytical Biochemistry, Vol. 222, pp. 389395, 1994), restriction fragment length polymorphism (RFLP) and hybridization assays. Other methods include high density arrays, mass spectrometry, molecular beacons, peptide nucleic acids, and mismatch cleavage based assays. These include but are not limited to bacteriophage T4 endonuclease VII (U.S. Pat. No. 6,110,684 issued Aug. 29, 2000; U.S. Pat. No. 6,183,958 issued Feb. 6, 2001, U.S. Pat. No. 5,958,692, U.S. Pat. No. 5,851,770, WO 00/18967 Apr. 6, 2000; WO 00/50639 Aug. 31, 2000) WO 00/18967). 5′ nucleases and/or 3′ exonucleases (U.S. Pat. No. 5,888,780, WO 98/50403A1, U.S. Pat. No. 5,719,028, WO 00/66607; WO056925) and others such as WO 073766, WO050871, WO 00/66607).

Techniques can also be classified as either target amplification or signal amplification. Target amplification involves the amplification (i.e., replication) of the target sequence to be detected, resulting in a significant increase in the number of target molecules. Target amplification strategies include the polymerase chain reaction (PCR), strand displacement amplification (SDA), and nucleic acid sequence based amplification (NASBA). Signal amplification strategies include the ligase chain reaction (LCR), cycling probe technology (CPT), invasive cleavage techniques such as Invader™ technology, Q-Beta replicase (QBR) technology, and the use of “amplification probes” such as “branched DNA” that result in multiple label probes binding to a single target sequence.

Further assays include, but are not limited to, ligation based assays, cleavage based assays (mismatch and invasive cleavage such as Invader™), and single base extension methods (see WO 92/15712, EP 0 371 437 B1, EP 0317 074 B1; Pastinen et al., Genome Res. 7: 606-614 (1997); Syvãnen, Clinica Chimica Acta 226: 225-236 (1994); and WO 91/13075).

The polymerase chain reaction (PCR) is widely used and described, and involves the use of primer extension combined with thermal cycling to amplify a target sequence; see U.S. Pat. Nos. 4,683,195 and 4,683,202, and PCR Essential Data, J. W. Wiley & sons, Ed. C. R. Newton, 1995, all of which are incorporated by reference. In addition, there are a number of variations of PCR which also find use in the invention, including “quantitative competitive PCR” or “QC-PCR”, “arbitrarily primed PCR” or “AP-PCR”, “immuno-PCR”, “Alu-PCR”, “PCR single strand conformational polymorphism” or “PCR-SSCP”, allelic PCR (see Newton et al. Nucl. Acid Res. 17: 2503 91989); “reverse transcriptase PCR” or “RT-PCR”, “biotin capture PCR”, “vectorette PCR”. “panhandle PCR”, and “PCR select cDNA subtraction”, Multiplex PCR amplification of SNP loci with subsequent hybridization to oligonucleotide arrays has been shown to be an accurate and reliable method of simultaneously genotyping at least hundreds of SNPs; see Wang et al., Science, 280: 1077 (1998); Schafer et al., Nature Biotechnology 16: 33-39 (1998).

Strand displacement amplification (SDA) is generally described in Walker et al., in Molecular Methods for Virus Detection, Academic Press, Inc., 1995, and U.S. Pat. Nos. 5,455,166 and 5,130,238, all of which are hereby incorporated by reference. Nucleic acid sequence based amplification (NASBA) is generally described in U.S. Pat. No. 5,409,818 and “Profiting from Gene-based Diagnostics”, CTB International Publishing Inc., N.J., 1996, both of which are incorporated by reference in their entirety.

Cycling probe technology (CPT) is a nucleic acid detection system based on signal or probe amplification rather than target amplification, such as is done in polymerase chain reactions. Cycling probe technology relies on a molar excess of labelled probe that contains a scissile linkage of RNA. Upon hybridization of the probe to the target, the resulting hybrid contains a portion of RNA: DNA. This area of RNA: DNA duplex is recognized by RNAse H and the RNA is excised, resulting in cleavage of the probe. The probe now consists of two smaller sequences which may be released, thus leaving the target intact for repeated rounds of the reaction. The unreacted probe is removed and the label is then detected. CPT is generally described in U.S. Pat. Nos. 5,011,769, 5,403,711, 5,660,988, and 4,876,187, and PCT published applications WO 95/05480, WO 95/1416, and WO 95/00667, all of which are specifically incorporated herein by reference. Invader™ technology is based on structure-specific polymerases that cleave nucleic acids in a site specific manner. Two probes are used: an “invader” probe and a “signalling” probe, that adjacent hybridize to a target sequence with a non-complementary overlap. The enzyme cleaves at the overlap due to its recognition of the “tail”, and releases the “tail” with a label. This can then be detected. The Invader technology is described in U.S. Pat. Nos. 5,846,717; 5,614,402; 5,719,028; 5,541,311; and 5,843,669, all of which are hereby incorporated by reference.

The oligonucleotide ligation assay (OLA), sometimes referred to as the ligation chain reaction (LCR)), involve the ligation of at least two smaller probes into a single long probe, using the target sequence as the template for the ligase. See generally U.S. Pat. Nos. 5,185,243, 5,679,524 and 5,573,907; EP 0 320 308 B1; EP 0 336 731 B1; EP 0 439 182 B1; WO 90/01069; WO 89/12696; and WO 89/09835.

“Rolling circle amplification” is based on extension of a circular probe that has hybridized to a target sequence. A polymerase is added that extends the probe sequence. As the circular probe has no terminus, the polymerase repeatedly extends the circular probe resulting in concatamers of the circular probe. As such, the probe is amplified. Rolling-circle amplification is generally described in Baner et al., (1998) Nuc. Acids Res. 26: 5073-5078; Barany, F. (1991) Proc. Natl. Acad. Sci. USA 88: 189-193; and Lizardi et al., (1998) Nat Genet. 19: 225-232, all of which are incorporated by reference in their entirety.

Branched DNA signal amplification (BDNA) relies on the synthesis of branched nucleic acids, containing a multiplicity of nucleic acid “arms” that function to increase the amount of label that can be put onto one probe. This technology is generally described in U.S. Pat. Nos. 5,681,702, 5,597,909, 5,545,730, 635,352, 5,594,118, 5,359,100, 5,124,246 and 5,681,697, all of which are hereby incorporated by reference. Similarly, dendrimers of nucleic acids serve to vastly increase the amount of label that can be added to a single molecule, using a similar idea but different compositions. This technology is as described in U.S. Pat. No. 5,175,270 and Nilsen et al., J. Theor. Biol. 187: 273 (1997), both of which are incorporated herein by reference.

Other methods include mismatch detection techniques using enzymatic cleavage such as resolvase (Variagenics resolvase, bacteriophage T4 endonuclease VII, U.S. Pat. No. 6,110,684, issued Aug. 29, 2000; U.S. Pat. No. 6,183,958, issued Feb. 6, 2001, U.S. Pat. No. 5,958,692, U.S. Pat. No. 5,851,770, WO 00/18967 Apr. 6, 2000; WO 00/50639 published Aug. 31, 2000) WO 00/18967). The use of 5′ nucleases and/or 3′ exonucleases for target dependent reactions using cleavage structures (Third Wave U.S. Pat. No. 5,888,780, WO 98/50403A1, U.S. Pat. No. 5,719,028, Aclara (WO 00/66607; WO056925). Orchid Biosciences (WO 073766, WO050871, WO 00/66607).

Screening Patients Having Low HDL-C or High Triglyceride Levels

ABC1 expression, biological activity, and mutational analysis can each serve as a diagnostic tool for low HDL or higher than normal triglyceride levels; thus determination of the genetic subtyping of the ABC1 gene sequence can be used to subtype low HDL or higher than normal triglyceride individuals or families to determine whether the low HDL or higher than normal triglyceride phenotype is related to ABC1 function. This diagnostic process can lead to the tailoring of drug treatments according to patient genotype, including prediction of side-effects upon administration of HDL increasing or triglyceride lowering drugs (referred to herein as pharmacogenomics). Pharmacogenomics allows for the selection of agents (e.g., drugs) for therapeutic or prophylactic treatment of an individual based on the genotype of the individual (e.g., the genotype of the individual is examined to determine the ability of the individual to respond to a particular agent).

Agents, or modulators which have a stimulatory or inhibitory effect on ABC1 biological activity or gene expression can be administered to individuals to treat disorders (e.g., cardiovascular disease, low HDL cholesterol, or a higher than normal triglyceride level) associated with aberrant ABC1 activity. In conjunction with such treatment, the pharmacogenomics (i.e., the study of the relationship between an individual's genotype and that individual's response to a foreign compound or drug) of the individual may be considered. Differences in efficacy of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of ABC1 protein, expression of ABC1 nucleic acid, or mutation content of ABC1 genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons (Eichelbaum, M., Clin. Exp. Pharmacol. Physiol., 23:983-985, 1996; Linder, M. W., Clin. Chem., 43:254-266, 1997). In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). Altered drug action may occur in a patient having a polymorphism (e.g., an single nucleotide polymorphism or SNP) in promoter, intronic, or exonic sequences of ABC1. Thus by determining the presence and prevalence of polymorphisms allow for prediction of a patient's response to a particular therapeutic agent. In particular, polymorphisms in the promoter region may be critical in determining the risk of HDL deficiency, higher than normal triglyceride level, and CVD.

In addition to the mutations in the ABC1 gene described herein, we have previously detected polymorphisms in the human ABC1 gene (PCT Publication WO 00/55318, filed Mar. 15, 2000; U.S. Utility application Ser. No. 09/526,193, filed Mar. 15, 2000; PCT Publication WO 00/0115676, filed Sep. 1, 2000; or U.S. Utility application Ser. No. 09/654,323, filed Sep. 1, 2000). These polymorphisms are located in promoter, intronic, and exonic sequence of ABC1. Using standard methods, such as direct sequencing, PCR, SSCP, or any other polymorphism-detection system, one could easily ascertain whether these polymorphisms are present in a patient prior to the establishment of a drug treatment regimen for a patient having low HDL, a higher than normal triglyceride level, cardiovascular disease, or any other ABC1-mediated condition. It is possible that some these polymorphisms are, in fact, weak mutations. Individuals harbouring such mutations may have an increased risk for cardiovascular disease; thus, these polymorphisms may also be useful in diagnostic assays.

Results

During the sequencing of 16 probands with TD or FHA^{9,10,14}, we have identified 12 SNPs in regions potentially involved in regulatory functions: 2 in the promoter, 4 in the 5′ UTR encoded by exons 1 and 2, and 6 within intron 1 (FIG. 1).

While sequencing, it became apparent that certain pairs of variants were in complete or near complete linkage disequilibrium with each other (FIG. 1, dashed lines). All individuals carrying the InsG319 variant (5 heterozygous, 2 homozygous) had the identical genotype at G378C, confirming a previous report of linkage disequilibrium between these two SNPs²¹. Of the 16 individuals sequenced, three individuals were heterozygous and 5 were homozygous for both the A-461C and A-362G variants; although, 1 individual was homozygous for the A-362G variant but did not carry the variant at −461. Finally, 2 of the 16 individuals were heterozygous and 5 homozygous for both the G-720A and G-1027A variants. Thus, for phenotypic analysis, only one variant of each of these pairs was analyzed.

Promoter Variants: The G-191C SNP is Associated with Increased and the C-17G with Decreased Coronary Events

We assessed the phenotypic effects of these SNPs in the REGRESS cohort, a well described cohort of Dutch men with proven CAD²². The more frequent allele of each variant in this cohort was designated A, while the less frequent allele was designated B. The carrier and B-allele frequencies of the SNPs are shown in Table 2.

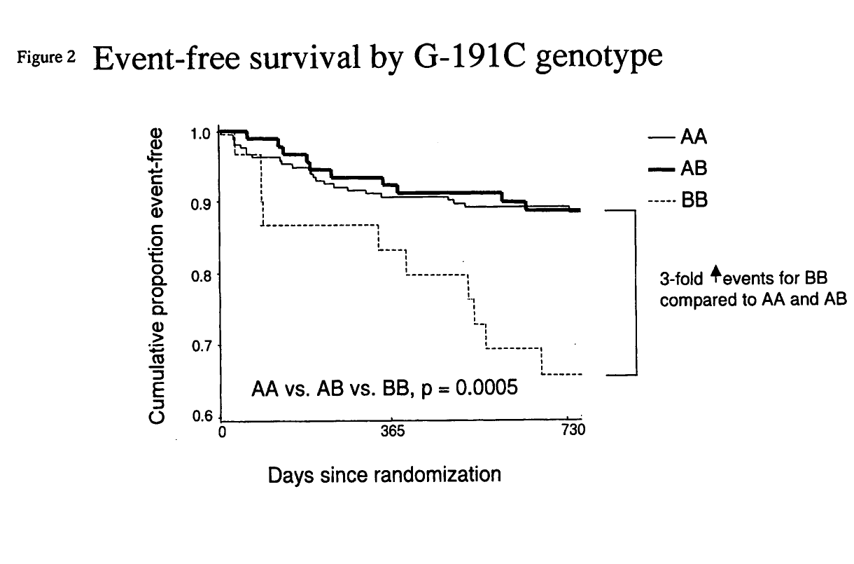

Individuals with the BB-genotype of the G-191C SNP had triple the incidence of coronary events during the two-year study compared to individuals with the AA-genotype (BB 33.3% (n=30) vs. AA 11.2% (n=214), p=0.001, Table 3), resulting in an odds ratio of 3.96 in BB individuals compared to AA (95% confidence interval 1.66-9.45, p=0.003), a similar magnitude as that observed in individuals heterozygous for ABC1 mutations¹⁴. This is illustrated in FIG. 2, which shows the cumulative proportion of individuals who remained event free during the study for each of the genotypes. The curve is significantly lower for BB's compared to AA's and AB's (p=0.0005 for AA vs. AB vs. BB, FIG. 2). In further support of the relationship of the B allele with an increased frequency of CAD, family members of BB-individuals had nearly twice the prevalence of an MI compared to non-carriers (BB 73.3% vs. AA 47.7%, p=0.01). The MSD, which measures diffuse atherosclerosis, was also indicative of increased atherosclerosis in BB's, although this did not reach statistical significance (Table 3). However, despite the differences in CAD, no significant differences in lipid levels between the genotypes were noted (Table 3).

To further confirm and replicate these findings, we genotyped this variant in a cohort of individuals with familial hypercholesterolemia available in the lab¹⁵ (and Clee et al, manuscript submitted). Within this cohort, carriers of the G-191C variant had increased vascular disease compared to non-carriers (14.8% of AA (n=110), 34.3% of AB (n=36), 26.1% of BB (n=48), p=0.03). But, as with the REGRESS cohort, no significant differences in TG (AA 1.50±0.75, AB 1.83±0.94, BB 1.44±0.80, p=0.11) or HDL-C (AA 1.29±0.44, AB 1.28±0.38, BB 1.20±0.25, p=0.41) amongst the genotypes were observed.

In contrast to the G-191C, carriers of the C-17G variant had fewer events during the trial than non-carriers (AB+BB 12.3% (n=351 vs. AA 18.2% (n=286, p=0.04, Table 4). Similarly, a smaller percentage of carriers had had an MI prior to the start of the study (AB+BB 43.6% vs. AA 52.8%, p=0.02). As with the G-191C, no significant differences in plasma lipid levels were observed between carriers and non-carriers.

This variant was also screened within our replication cohort, and similar results were obtained. Homozygous (BB) carriers of this variant had greater than 3.5-fold fewer events than non-carriers (26.0% of AA (n=80) vs. 7.1% of BB (n=14), p=0.18), although this did not reach statistical significance. Again, no significant differences in TG (AA 1.58±0.91, AB 1.40±0.59 (n=28), BB 1.59±0.55, p=0.61) or HDL-C (AA 1.22±0.34, AB 1.30±0.41, BB 1.31±0.55, p=0.56) were observed between the genotypes.

SNPs in the 5′UTR: The C69T SNP is Associated with Increased and the InsG319 with Decreased Atherosclerosis

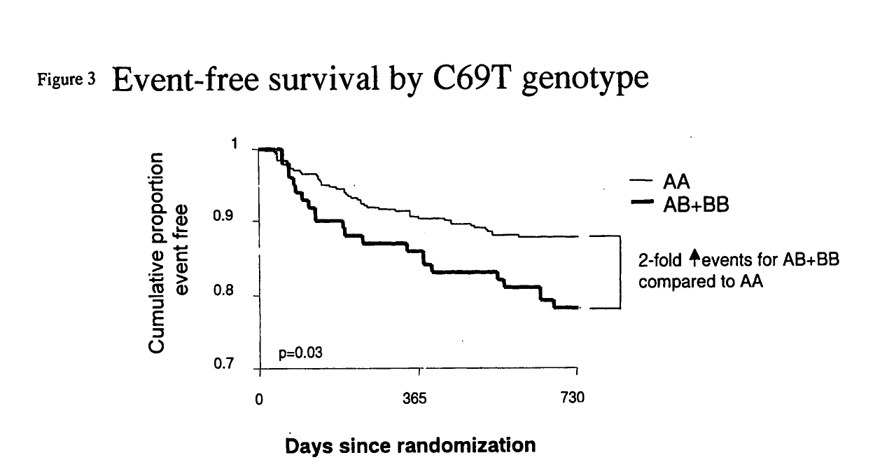

Carriers (AB+BB) of the C69T SNP had approximately twice the number of coronary events during the trial as non-carriers (AA; p=0.03, Table 5). This difference is illustrated in the event-free survival curve (p=0.03, FIG. 3). Within the placebo group, carriers of the C69T variant had increased progression of diffuse atherosclerosis (delta-MSD) compared to non-carriers (AA vs. AB+BB, p=0.01, Table 5). We observed a similar trend for the progression of focal atherosclerosis in the placebo group (p=0.11, Table 5). No difference in progression of atherosclerosis was seen for the group treated with pravastatin, or the whole group, which suggests that pravastatin may be able to overcome the effects of this variant. As with the promoter variants, no differences in mean lipid levels were observed in carriers of this variant compared to non-carriers (Table 5).

Increased vascular disease was also observed in carriers of this variant in our replication cohort (18.8% of AA (n=192) vs. 32.2% of AB+BB (n=59), p=0.046; OR=2.05 (95% Cl 1.06-3.96). Once again, no significant differences in lipid levels were observed between carriers and non-carriers of this variant within the replication group (TG: 1.59±0.85 vs. 1.62±0.75, p=0.84; HDL: 1.28±0.37 vs. 1.23±0.35, p=0.38; for AA vs. AB+BB, respectively).

In REGRESS, carriers of the C117G SNP had a gene-dose-dependent increase of TG (AA 1.82±0.79, AB 2.69±0.40, BB 2.77±0.26, p=0.003 for AA vs. BB, Table 4) compared to non-carriers. No other differences in CAD or lipid levels were observed in C117G carriers. However, this trend was not maintained within the replication cohort (TG: 1.63±0.86, n=258 1.30±0.48, n=9 AA vs. AB+BB, p=0.08), although the number of carriers identified in this cohort was small. No significant differences in HDL-C or vascular disease were observed in this cohort (data not shown).

Linkage disequilibrium between the InsG319 and G378C SNPs was confirmed in the REGRESS population, by screening a subset of individuals for both variants. The InsG319 and the G378C variants were in almost complete linkage disequilibrium (D′=0.90, p=0.13): of the 59 individuals that were screened for both these variants, the genotype of only 1 individual was discordant (AA for InsG319 and AB for G378C). The remainder (41 AA, 15 AB, 2 BB) were identical at both loci. The entire REGRESS cohort was thus genotyped for only the InsG319.

Carriers of this variant have less focal atherosclerosis (MOD: AA 1.76±0.36 vs. AB 1.82±0.34 vs. BB 1.94±0.32, p=0.05, Table 4) and less diffuse atherosclerosis (MSD: AA 2.71±0.37 vs. AB 2.84±0.33 vs. BB 3.13±0.56, p<0.001) compared to non-carriers. No differences in events or mean lipid levels were observed in either the REGRESS or replication cohort (n=386 AA, 120 AB, 7 BB; data not shown).

Intronic SNPs

Homozygous carriers of the A-1095G SNP had an increased progression of focal atherosclerosis was significantly higher in BB's than in AA's in the placebo group (delta-MOD: AA 0.13±0.22 (n=216) vs. BB 0.42±1.00 (n=5), p=0.01). This trend was not observed in the pravastatin group (delta-MOD: AA 0.07±0.26 (n=222) vs. BB −0.05±0.18 (n=7), p=0.22). Furthermore, the history of CAD in their families was increased compared to AA-individuals (AA 50.1% vs. BB 83.3%, p=0.02). Consistent with this, trends towards decreased HDL-C (AA 0.93±0.24 vs. BB 0.83±0.20, p=0.15) and increased TG (AA 1.78±0.76 vs. BB 2.18±0.78, p=0.07) were observed in homozygous carriers compared to non-carriers (Table 4). Similar trends were observed in homozygous carriers within the replication cohort. BB individuals (n=12) had increased vascular disease (50% vs. 20.5% in AA's (n=327), p=0.03; OR=3.88, 95% Cl 1.21-12.42), and although TG were increased (1.71±0.96 vs. 1.64±0.85) and HDL-C decreased (1.15±0.33 vs. 1.26±0.33), neither finding was statistically significant (p=0.81, 0.29, respectively).

No significant differences in CAD or lipid levels were observed in carriers of the InsCCCT-1163, the G-720A (and G-1027A) or the A-362G (and A-461C)SNPs (Table 5). As observed during sequencing, the G-1027A variant was in near complete linkage disequilibrium with the G-720A variant (D′=0.84, p=0.02) in the subset of REGRESS screened for both: all 15 individuals that were BB for G-1027A were also BB for G-720A, and of the 31 individuals that were AB for the G-1027A, 30 individuals were also AB for G-720A; the other was BB. Linkage disequilibrium between the A-461C and the A-362G variants was also confirmed in REGRESS (D′=0.76, p=0.06): all individuals screened for both these variants (n=52) had the same genotype for both the variants (n=29 AB, 6 BB).

Partial Linkage Disequilibrium Between SNPs does not Alter the Phenotypic Effects of each SNP

In addition to the near complete linkage disequilibrium already described, partial linkage disequilibrium between variants may also exist, and thus the phenotype attributed to one variant may be partially accounted for by its association with another functional variant in some of the carriers. We have addressed the issue of potential partial linkage disequilibrium between the SNPs by examining their pair-wise associations (Table 6 and see ref. 15). Of the SNPs that were associated with functional effects, 3 pairs were significantly associated with each other: the C69T with the G-191C, the C17G with the C117G, and the A-1095G with the InsG319. We therefore examined each of these pairs in more detail.

Nearly half (45%) of the G-191C carriers were also carriers of the C69T. To examine whether the functional effects of the G-191C are independent of the C69T, we examined the G-191C in the subgroup of individuals who were all non-carriers (i.e. AA) of the C69T (G-191C: AA n=200, AB n=48, BB n=15). Statistical analysis after excluding carriers of the C69T variant yielded similar results compared to the analysis without this correction. Coronary events were still increased approximately 3-fold in homozygous carriers of the G-191C (AA 11%, AB 10.4%, BB 33.3%, p=0.01 for AA vs. BB), as was a family history of CAD (AA 49.5%, AB 39.6%, BB 73.3%, p=0.08 for AA vs. BB). Furthermore, no differences in plasma lipid levels were unmasked by the exclusion of C69T carriers. This suggests that the functional effects attributed to the G-191C variant are not due to effect of the C69T variant.

Unfortunately, as 87% of C69T carriers also have the G-191C variant, too few carriers of the C69T variant were left (AB+BB n=8) for reasonable statistical power after exclusion of G-191C carriers. Therefore, it is uncertain whether the effects ascribed to the C69T are due to the G-191C.

A smaller percentage of C-17G carriers were also carriers of the C117G (17%). We performed statistical analysis for the C-17G SNP in the subgroup of individuals without the C17G variant (C-17G: AA 215, AB 205, BB 28). Similar results were obtained as for the whole group: events were reduced (11.6 vs. 18.6%, p=0.04), as were MIs prior to the trial (44.2 vs. 53.0%, p=0.06) for AB+BB compared to AA. Thus, the functional effects described for the C-17G SNP are not due to the C117G. Still, no significant differences in plasma lipid levels were observed.

Of the carriers of C117G, 82% were also carriers of C-17G. Thus, selection of carriers of C17G who did not have the C-17G variant did not result in enough carriers to perform statistical analysis. However, as carriers of the C-17G do not show any differences in plasma TG levels, these trends observed for carriers of the C117G are unlikely to be due to the co-presence of the C-17G SNP.

As all carriers of the InsG319 were carriers of the A-1095G, we could not select a subgroup of InsG319 carriers who did not have the A-1095G variant. However, to address if the phenotype of the InsG319 variant is independent of the A-1095G variant, we performed statistical analysis on carriers of the InsG319 variant compared to non-carriers, in the subgroup of individuals who were carriers (AB's and BB's) of the A-1095G variant (InsG319: AA n=53 and AB+BB n=87). Keeping carrier status for the A-1095G constant, there was still significantly less diffuse atherosclerosis (2.86±0.35 vs. 2.70±0.37, p=0.01) and a trend towards less focal atherosclerosis (1.84±0.35 vs. 1.76±0.33, p=0.18) in carriers of the InsG319 (AB+BB) compared to non-carriers (AA). No significant differences in lipid levels were observed, although there were mild trends towards higher HDL-C (0.93±0.24 vs. 0.88±0.17, p=0.19) and lower TG (1.66±0.69 vs. 1.84±0.85, p=0.17) between these groups. This suggests that atherosclerosis is reduced in InsG319 carriers, independent of the A-1095G variant.

Sixty-three percent of the carriers of A-1095G were also carriers of InsG319. Following exclusion of the InsG319 carriers, no A-1095G BB individuals (in whom the phenotype was observed) remained for analysis. However, as the InsG319 was not associated with alterations in plasma lipid levels and was associated with an opposite effect on vascular disease, the effects of the A-1095G are unlikely to be due to the InsG319.

Here we present an analysis of 12 non-coding SNPs in the promoter, intron 1 and 5′UTR of the ABC1 gene. We report that several of these common variants are associated with altered severity of arteriosclerosis, without any observed changes in plasma lipid levels (summarized in Table 7).

The G-191C SNP, independent of the C69T SNP with which it is in partial linkage disequilibrium, was associated with an approximately 3-fold increase in coronary events, resulting in an odds ratio similar to that of individuals heterozygous for mutations in ABC1. In further support of this was a significantly increased family history of CAD. Increased vascular disease was also observed in our replication cohort, but in neither cohort were significant differences in plasma lipid levels observed.

Similarly, the C69T SNP was also associated with increased coronary events and increased atherosclerotic progression, again with no differences in plasma lipid levels. These findings were also observed in our replication cohort. Although this variant is in partial linkage disequilibrium with the G-191C SNP that had similar effects, the C69T variant was associated with increased events in both homozygous and heterozygous carriers of the variant, the majority of whom were heterozygous for the G-191C, whereas the G-191C SNP only showed increased events in homozygous carriers in REGRESS. Thus, the effects of the C69T are not likely to be due entirely to the G-191C SNP.

In contrast, both the C-17G and InsG319 SNPs were associated with reduced arteriosclerosis. The C-17G SNP was associated with a reduction in coronary events both during and prior to the REGRESS study, and with a 3.5-fold reduction in vascular disease events in the replication cohort. The InsG319 SNP was associated with reduced focal and diffuse arteriosclerosis. These effects were independent of other SNPs found in partial linkage disequilibrium with these variants. As with the G-191C and C69T SNPs, no significant differences in plasma lipid levels were observed in carriers of either variant.

Thus, several ABC1 regulatory variants were associated with an altered risk of CAD but without corresponding differences in lipid levels. These findings suggest that decreases or increases in RCT activity may change the net flux of cholesterol from the vessel wall towards the liver, without altering plasma lipid levels. Thus, ABC1 variation may directly influence the atherosclerotic process without altering plasma lipid levels. One explanation for these findings might be that only larger changes in efflux result in measurable changes in plasma lipid levels, whereas smaller changes might still directly impact cholesterol accumulation within the vessel wall. Indeed, cholesterol efflux is highly correlated to vessel wall intima-media thickness (van Dam et al, manuscript in preparation). Alternatively, these variants may influence ABC1 regulation in certain tissues (e.g. macrophages) or under some environmental stimuli (e.g. in response to cholesterol loading or other atherogenic stimuli) but not others, and thus may directly influence ABC1 activity within the vessel wall but not elsewhere. Furthermore, lesion macrophages likely constitute a small percentage of total body cells eliminating excess cholesterol and contributing to plasma HDL-C levels, and thus changes in macrophage ABC1 activity may not directly result in changes in plasma HDL-C levels. Therefore alterations in ABC1 regulation may impact cholesterol accumulation specifically in the vessel wall without changing plasma lipid levels.

The A-1095G SNP was also associated with more progression of focal arteriosclerosis and more CAD in family members of the REGRESS participants, and with increased vascular disease events in the replication cohort. In both cohorts there were mild trends towards decreased HDL-C and increased TG in carriers, however in neither case were they significant. Therefore, this variant may either exert a very mild effect, or no effect on plasma lipid levels, again suggesting that ABC1 regulatory variants may have a significant influence on CAD without obvious changes in plasma lipid levels.

The C117G SNP was the only SNP directly associated with altered plasma lipid levels, being associated with increased TG levels in the REGRESS cohort. However, these findings were not observed within the small number of carriers in the replication cohort. Thus it is uncertain whether this variant truly influences plasma TG levels. Analysis in additional cohorts will be required to ascertain whether this SNP affects plasma lipid levels.

The precise mechanism behind the regulatory function of these SNPs will require further analysis. It is possible that the nucleotide substitutions directly alter transcription factor binding sites, thus influencing the transcription of ABC1. It is also possible that transcription occurs at a normal rate from the two alleles, but that mRNA stability is altered or that the two allelic forms of the mRNA possess different abilities to initiate translation, perhaps as a result of differences in secondary structure²⁵. The SNPs may also influence splice sites, their recognition, or splicing enhancers²⁶. SNPs within non-coding regions have previously been shown to have such functional effects^{16-18,26-28}. To understand the mechanism by which these SNPs influence ABC1 expression, they will need to be re-created in vitro and their functionality at basal levels and in response to various regulatory stimuli in various cell types assessed.

This is the first report describing the in vivo effects of regulatory variants within ABC1, and demonstrating that these variants have significant effects on the population risk of CAD. Here, we have shown that several common SNPs in non-coding regions of the ABC1 gene are associated with an altered risk of CAD in the absence of detectable changes in plasma lipids.

These findings suggest that proper regulation of ABC1 is critical for RCT and thus prevention of atherosclerotic vascular disease. As described previously, identification of polymorphisms in the non-coding region of ABCA1 can provide valuable information for predictive diagnosis of cardiovascular and other disorders and diseases. Based on the disclosure herein, those skilled in the art can develop nucleic acid sequencing/analysis compositions methods and kits that are suitable for diagnosis of these diseases. Any method of determining the target gene sequence can be used in the method of this invention, including full length or partial gene sequencing, probe based assays, RFLP and all other techniques known to those in the art.

Furthermore, sequence analysis of the non-coding region of ABCA1 can also be used to predict drug responsiveness, susceptibility to side-effects of drugs, and, importantly, it is useful for designing clinical trials, as generally encompassed by the concept of pharmacogenetics. The polymorphisms or mutations disclosed herein can be correlated to a patient response database in order to generate a prognostic database for aiding selection of an appropriate therapeutic regime for a patient. Single nucleotide polymorphisms (SNPs) in ABCA1 are related to drug responsiveness, drug side effects, and are implicated in diseases and disorders disclosed in this invention.

Clinical trials for therapeutic agents for treatment of cardiovascular and other diseases and disorders can be simplified and made more accurate by performing sequence analysis of the non-coding region of ABCA1 as identified herein. In one embodiment, patients enrolled in a clinical trial for a new therapeutic agent give a tissue sample, and the nucleic acid sequence of the non-coding region of ABCA1 is determined. Patients are categorized by their particular genetic variant and their response to the therapeutic agent. A correlation between drug responsiveness and genetic variant may be determined. This correlation then becomes an important tool for physicians who prescribe the drug; all patients who are indicated for the drug are first typed for the genetic variant to ensure that they will have the desired clinical outcome.

Alternatively, clinical trial design may be improved by pre-selecting patients who are likely to have positive outcomes to a therapeutic agent based on their having preferred genetic variants of the therapeutic targets disclosed herein. All potential patients are first sequenced at the relevant target gene, and only those that have the preferred variant are enrolled in the trial. This technique will greatly reduce the number of patients that are required in a clinical trial to determine efficacy of the therapeutic agent.

In accordance with the foregoing, the present invention also contemplates a method for identifying a therapeutic agent for administration to a patient in need thereof, comprising comparing a nucleotide sequence of a non-coding region of an ABCA1 gene of said patient to a database (such as where the database comprises ABCA1 nucleotide sequences comprising the polymorphic sequences disclosed in Table 1) that correlates nucleic acid sequences of ABCA1 genes with the effectiveness of therapeutic agents in beneficially regulating lipid levels in a patient, thereby identifying a therapeutic agent for administration to said patient.

Further, the present invention provides a method for identifying a candidate for enrolment in a program of clinical trials of a potential therapeutic agent, comprising comparing a nucleotide sequence of a non-coding region of an ABCA1 gene of said candidate to a database that correlates nucleic acid sequences of ABCA1 genes with the effectiveness of therapeutic agents in beneficially regulating lipid levels in a patient, thereby identifying a candidate for enrolment in a program of clinical trials, again especially where the database comprises ABCA1 nucleotide sequences comprising the polymorphic sequences disclosed in Table 1.

The present invention also relates to a process that comprises a method for producing a product comprising identifying an agent according to one of the disclosed processes for identifying such an agent (i.e., the therapeutic agents identified according to the assay procedures disclosed herein) wherein said product is the data collected with respect to said agent as a result of said identification process, or assay, and wherein said data is sufficient to convey the chemical character and/or structure and/or properties of said agent. For example, the present invention specifically contemplates a situation whereby a user of an assay of the invention may use the assay to screen for compounds having the desired enzyme modulating activity and, having identified the compound, then conveys that information (i.e., information as to structure, dosage, etc) to another user who then utilizes the information to reproduce the agent and administer it for therapeutic or research purposes according to the invention. For example, the user of the assay (user 1) may screen a number of test compounds without knowing the structure or identity of the compounds (such as where a number of code numbers are used the first user is simply given samples labeled with said code numbers) and, after performing the screening process, using one or more assay processes of the present invention, then imparts to a second user (user 2), verbally or in writing or some equivalent fashion, sufficient information to identify the compounds having a particular modulating activity (for example, the code number with the corresponding results). This transmission of information from user 1 to user 2 is specifically contemplated by the present invention.

All publications mentioned in this specification are herein incorporated by reference to the same extent as if each independent publication was specifically and individually indicated to be incorporated by reference.

While the invention has been described in connection with specific embodiments thereof, it will be understood that it is capable of further modifications. This application is intended to cover any variations, uses, or adaptations following, in general, the principles of the invention and including such departures from the present disclosure within known or customary practice within the art to which the invention pertains and may be applied to the essential features hereinabove set forth.

TABLE 1
Methods for screening non-coding ABCA1 SNPs

| | | | | | Products (bp): |
|---|---|---|---|---|---|
| | Forward oligo (5′-->3′)^{a} | “A” allele | Annealing | | wt “A” allele |
| Variant | Reverse oligo (5′-->3′)^{a} | “B” allele | temp. (C.) | Enzyme | variant “B” allele |
| G-191C | CAGCGCTTCCCGCGCGTCTTAG | G | 60 | HgaI | 287, 55, 3 |
| | (SEQ ID NO: 1) | | | | |
| | CCACTCACTCTCGTCCGCAATTAC | C | | | 342, 3 |
| | (SEQ ID NO: 2) | | | | |
| C-17G | CTGCTGAGTGACTGAACTACATAAACAGAGGCCGGGTA | C | 60 | Rsa I | 161 |
| | (SEQ ID NO: 3) | | | | |
| | CCACTCACTCTCGTCCGCAATTAC | G | | | 124, 37 |
| | (SEQ ID NO: 4) | | | | |
| C69T | CAGCGCTTCCCGCGCGTCTTAG | C | 60 | BsmAI | 345 |
| | (SEQ ID NO: 5) | | | | |
| | CCACTCACTCTCGTCCGCAATTAC | T | | | 310, 35 |
| | (SEQ ID NO: 6) | | | | |
| C117G | CTGGCTTTCTGCTGAGTGAC | C | 60 | Eco 0109 | 284, 175 |
| | (SEQ ID NO: 7) | | | I | |
| | GATCAAAGTCCCCGAAACC | G | | | 459 |
| | (SEQ ID NO: 8) | | | | |
| A-362G | ACTCAGTTGTATAACCCACTGAAAATTAGT | A | 55 | Mbo II | 224, 26 |
| | (SEQ ID NO: 9) | | | | |
| | TTCTATAGATGTTATCATCTGGG | G | | | 134, 90, 26 |
| | (SEQ ID NO: 10) | | | | |
| A-461C | ACTCAGTTGTATAACCCACTGAAAATGAGT | A | 55 | Hinf I | 150, 100 |
| | (SEQ ID NO: 11) | | | | |
| | TTCTATAGATGTTATCATCTGGG | C | | | 123, 100, 27 |
| | (SEQ ID NO: 12) | | | | |
| G-720A | TCATCTAAGGCACGTTGTGG | G | 60 | Hpa II | 450 |
| | (SEQ ID NO: 13) | | | | |
| | CCTCAAGCCTGGAGTGACTT | A | | | 306, 144 |
| | (SEQ ID NO: 14) | | | | |
| G-1027A | ATGGCAAACAGTCCTCCAAG | G | 60 | Nco I | 170, 41 |
| | (SEQ ID NO: 15) | | | | |
| | ACCCTAGCGCTGTGTCTCTG | A | | | 105, 65, 41 |
| | (SEQ ID NO: 16) | | | | |
| A-1095G | ATGGCAAACAGTCCTCCAAG | A | 60 | MspA1 I | 211 |
| | (SEQ ID NO: 17) | | | | |
| | ACCCTAGCGCTGTGTCTCTG | G | | | 172, 39 |
| | (SEQ ID NO: 18) | | | | |
| | TGTGTGTCCTCCCTTCCATT | no ins | 60 | Mnl I | 144, 28, 11, 4 |
| | (SEQ ID NO: 19) | | | | |
| insCCCT-1163 | CTTGGAGGACTGTTTGCCAT | ins | | | 100, 48, 28, |
| | (SEQ ID NO: 20) | CCCT | | | 11, 4 |
| insG319 | CCCCTCCTGCTTTATCTTTCAGTTAATGACCAGCCCCG | no ins | 55 | Sma I | 246 |
| | (SEQ ID NO: 21) | | | | |
| | ATCCCCAACTCAAAACCACA | ins G | | | 210, 37 |
| | (SEQ ID NO: 22) | | | | |
| G378C | GCCGCTGCCTTCCAGGGCTCCCGAGCCACACGCTGCG | G | 55 | Aci I | 108, 41, 33, 5 |
| | (SEQ ID NO: 23) | | | | |
| | ATCCCCAACTCAAAACCACA | C | | | 141, 41, 5 |
| | (SEQ ID NO: 24) | | | | |

TABLE 2
Frequencies of ABCA1 SNPs in the REGRESS Population.

| | | |
|---|---|---|
| Nucleotide | “B” | Frequency |

| Change | allele | carrier (%) | B-allele | N^{a} |
|---|---|---|---|---|
| Promoter | | | | |
| G-191C | C | 35.9 | 0.225 | 668 |
| C-17G | G | 55.1 | 0.323 | 1274 |
| 5′ UTR | | | | |
| C69T | T | 20.4 | 0.138 | 812 |
| C117G | G | 11.9 | 0.065 | 1096 |
| Ins G319 | Ins | 16.2 | 0.085 | 1396 |
| Intron 1 | | | | |
| Ins CCCT-1163 | Ins | 2.1 | 0.011 | 1232 |
| A-1095G | G | 28.2 | 0.151 | 1220 |
| G-720A | A | 68.8 | 0.448 | 910 |
| A-362G | G | 65.5 | 0.445 | 1060 |

^{a}N refers to the number of alleles screened.

TABLE 3
G-191C carriers compared to non-carriers

| | | | | P-value | | |
|---|---|---|---|---|---|---|
| | | | | AA vs. | AA vs. | AA vs. AB vs. |
| | AA | AB | BB | BB | AB + BB | BB |
| n | 214 | 90 | 30 | | | |
| age | 56.6 ± 8.0 | 59.8 ± 8.8 | 55.3 ± 8.2 | 0.41 | 0.07 | 0.18 |
| Total Cholesterol | 5.98 ± 0.87 | 6.17 ± 0.90 | 6.12 ± 1.10 | 0.43 | 0.08 | 0.22 |
| LDL Cholesterol | 4.24 ± 0.80 | 4.45 ± 0.82 | 4.29 ± 0.91 | 0.75 | 0.07 | 0.14 |
| HDL Cholesterol | 0.93 ± 0.23 | 0.92 ± 0.22 | 0.94 ± 0.29 | 0.83 | 0.90 | 0.89 |
| Triglycerides | 1.79 ± 0.73 | 1.78 ± 0.74 | 1.95 ± 0.77 | 0.27 | 0.73 | 0.51 |
| MSD | 2.71 ± 0.39 | 2.73 ± 0.40 | 2.65 ± 0.34 | 0.19 | 0.96 | 0.69 |
| MOD | 1.74 ± 0.35 | 1.75 ± 0.33 | 1.76 ± 0.30 | | 0.83 | 0.96 |
| events % (n) | 11.2 (24) | 11.1 (10) | 33.3 (10) | 0.001 | 0.39 | 0.003 |
| family history CAD % (n) | 47.7 (102) | 43.1 (31) | 73.3 (22) | 0.01 | 0.01 | 0.02 |

TABLE 4
Lipid Levels and CAD in Carriers of ABCA1 SNPs.

| | | | | P-values | | |
|---|---|---|---|---|---|---|
| | | | | | AA vs. | AA vs. AB |
| | AA | AB | BB | AA vs. BB | AB + BB | vs. BB |
| C-17G | | | | | | |
| n | 286 | 290 | 61 | | | |
| HDL | 0.92 ± 0.22 | 0.93 ± 0.23 | 0.88 ± 0.21 | 0.19 | 0.91 | 0.33 |
| TG | 1.85 ± 0.75 | 1.77 ± 0.81 | 1.98 ± 0.82 | 0.23 | 0.52 | 0.14 |
| MOD | 1.77 ± 0.37 | 1.78 ± 0.35 | 1.72 ± 0.35 | 0.33 | 0.86 | 0.55 |
| MSD | 2.75 ± 0.39 | 2.74 ± 0.38 | 2.65 ± 0.39 | 0.07 | 0.48 | 0.22 |
| events % (n) | 18.2 (52) | 12.4 (36) | 11.5 (7) | 0.21 | 0.04 | 0.11 |
| C117G | | | | | | |
| n | 487 | 59 | 6 | | | |
| HDL | 0.92 ± 0.23 | 0.96 ± 0.25 | 0.95 ± 0.17 | 0.32 | 0.25 | 0.51 |
| TG | 1.82 ± 0.79 | 2.69 ± 0.40 | 2.77 ± 0.26 | <0.0001 | 0.45 | 0.67 |
| MOD | 1.75 ± 0.36 | 1.81 ± 0.40 | 1.70 ± 0.15 | 0.28 | 0.32 | 0.48 |
| MSD | 2.74 ± 0.38 | 2.69 ± 0.40 | 2.77 ± 0.26 | 0.55 | 0.45 | 0.67 |
| events % (n) | 14.8 (72) | 8.5 (5) | 33.3 (2) | 0.21 | 0.5 | 0.19 |
| InsG319 | | | | | | |
| n | 581 | 107 | 6 | | | |
| HDL | 0.92 ± 0.23 | 0.93 ± 0.23 | 0.89 ± 0.30 | 0.75 | 0.88 | 0.76 |
| TG | 1.80 ± 0.77 | 1.68 ± 0.66 | 1.89 ± 0.84 | 0.78 | 0.29 | 0.16 |
| MOD | 1.76 ± 0.36 | 1.82 ± 0.34 | 1.94 ± 0.32 | 0.22 | 0.11 | 0.05 |
| MSD | 2.71 ± 0.37 | 2.84 ± 0.33 | 3.13 ± 0.56 | 0.01 | <0.001 | <0.001 |
| events % (n) | 15.5 (90) | 13.1 (14) | 16.7 (1) | 0.94 | 0.81 | 0.55 |
| InsCCCT-1163 | | | | | | |
| n | 603 | 12 | 1 | | | |
| HDL | 0.93 ± 0.23 | 0.85 ± 0.18 | 0.82 | 0.63 | 0.21 | 0.46 |
| TG | 1.76 ± 0.76 | 1.83 ± 0.81 | 1.67 | 0.90 | 0.81 | 0.95 |
| MOD | 1.76 ± 0.36 | 1.82 ± 0.31 | 1.76 | 1.00 | 0.58 | 0.85 |
| MSD | 2.74 ± 0.37 | 2.83 ± 0.38 | 2.34 | 0.28 | 0.57 | 0.37 |
| events % (n) | 14.9 (90) | 8.3 (1) | 100 (1) | 0.02 | 0.96 | 0.05 |
| A-1095G | | | | | | |
| n | 438 | 160 | 12 | | | |
| HDL | 0.93 ± 0.24 | 0.93 ± 0.22 | 0.83 ± 0.20 | 0.15 | 0.53 | 0.28 |
| TG | 1.78 ± 0.76 | 1.74 ± 0.75 | 2.18 ± 0.78 | 0.07 | 0.88 | 0.15 |
| MOD | 1.76 ± 0.37 | 1.79 ± 0.34 | 1.79 ± 0.41 | 0.78 | 0.28 | 0.56 |
| MSD | 2.71 ± 0.38 | 2.76 ± 0.35 | 2.98 ± 0.57 | 0.02 | 0.05 | 0.03 |
| events % (n) | 13.7 (60) | 14.4 (23) | 16.7 (2) | 0.77 | 0.8 | 0.94 |
| G-720A | | | | | | |
| n | 142 | 218 | 95 | | | |
| HDL | 0.89 ± 0.20 | 0.92 ± 0.22 | 0.91 ± 0.23 | 0.48 | 0.31 | 0.52 |
| TG | 1.87 ± 0.77 | 1.86 ± 0.77 | 1.84 ± 0.80 | 0.77 | 0.78 | 0.96 |
| MOD | 1.76 ± 0.35 | 1.75 ± 0.37 | 1.70 ± 0.33 | 0.19 | 0.46 | 0.43 |
| MSD | 2.72 ± 0.38 | 2.72 ± 0.35 | 2.69 ± 0.38 | 0.55 | 0.85 | 0.77 |
| events % (n) | 12.7 (18) | 14.2 (31) | 12.6 (12) | 0.99 | 0.88 | 0.89 |
| A-362G | | | | | | |
| n | 182 | 221 | 124 | | | |
| HDL | 0.94 ± 0.23 | 0.94 ± 0.24 | 0.93 ± 0.2 | 0.82 | 0.91 | 0.97 |
| TG | 1.86 ± 0.82 | 1.72 ± 0.77 | 1.86 ± 0.76 | 0.94 | 0.22 | 0.11 |
| MOD | 1.75 ± 0.35 | 1.76 ± 0.35 | 1.74 ± 0.35 | 0.98 | 0.80 | 0.90 |
| MSD | 2.69 ± 0.40 | 2.74 ± 0.37 | 2.73 ± 0.37 | 0.38 | 0.21 | 0.45 |
| events % (n) | 16.5 (30) | 13.6 (30) | 11.3 (14) | 0.20 | 0.24 | 0.42 |

TABLE 5
C69T Carriers Compared to Non-Carriers

| | | | | P-value | | |
|---|---|---|---|---|---|---|
| | | | | | AA vs. | AA vs. AB vs. |
| | AA | AB | BB | AA vs. BB | AB + BB | BB |
| n | 323 | 54 | 29 | | | |
| age | 56.1 ± 8.2 | 55.0 ± 8.0 | 55.7 ± 6.6 | 0.80 | 0.40 | 0.66 |
| Total Cholesterol | 6.01 ± 0.87 | 6.15 ± 0.88 | 6.19 ± 1.04 | 0.29 | 0.17 | 0.38 |
| LDL Cholesterol | 4.28 ± 0.79 | 4.43 ± 0.78 | 4.34 ± 0.96 | 0.70 | 0.23 | 0.43 |
| HDL Cholesterol | 0.91 ± 0.22 | 0.91 ± 0.19 | 0.95 ± 0.30 | 0.36 | 0.79 | 0.70 |
| Triglycerides | 1.82 ± 0.75 | 1.80 ± 0.76 | 2.00 ± 0.80 | 0.22 | 0.61 | 0.44 |
| MSD | 2.72 ± 0.39 | 2.68 ± 0.37 | 2.70 ± 0.33 | 0.79 | 0.48 | 0.76 |
| MOD | 1.74 ± 0.34 | 1.70 ± 0.36 | 1.85 ± 0.29 | 0.09 | 0.86 | 0.16 |
| events %(n) | 12.4 (40) | 20.4 (11) | 24.1 (7) | 0.07 | 0.03 | 0.09 |
| Placebo | | | | | | |
| n | 159 | 26 | 17 | | | |
| delta-MSD | 0.10 ± 0.21 | 0.21 ± 0.29 | 0.22 ± 0.21 | 0.03 | 0.01 | 0.02 |
| delta MOD | 0.13 ± 0.27 | 0.19 ± 0.31 | 0.25 ± 0.26 | 0.08 | 0.11 | 0.25 |
| events % (n) | 16.4 (26) | 26.9 (7) | 35.3 (6) | 0.05 | 0.04 | 0.11 |
| Pravastatin | | | | | | |
| n | 64 | 28 | 12 | | | |
| delta-MSD | 0.08 ± 0.19 | 0.05 ± 0.13 | −0.02 ± 0.10 | 0.08 | 0.16 | 0.23 |
| delta-MOD | 0.07 ± 0.21 | 0.27 ± 0.97 | −0.05 ± 0.15 | 0.06 | 0.28 | 0.11 |
| events % (n) | 8.5 (14) | 14.3 (4) | 8.3 (1) | 0.28 | 0.23 | 0.51 |

TABLE 6
P-Values for Pairwise Associations of the ABCA1 SNPs in REGRESS

| | | | | | insCCCT- | | | | |
|---|---|---|---|---|---|---|---|---|---|
| | G-191C | C-17G | C69T | C117G | 1163 | A-1095G | G-720A | A-362G | insG319 |
| G-191C | — | | | | | | | | |
| C-17G | NA | — | | | | | | | |
| C69T | <0.0001 | NA | — | | | | | | |
| C117G | NA | <0.0001 | NA | — | | | | | |
| insCCCT- | NA | NA | NA | NA | — | | | | |
| 1163 | | | | | | | | | |
| A-1095G | NA | NA | NA | NA | <0.0001 | — | | | |
| G-720A | NA | NA | NA | NA | NA | NA | — | | |
| A-362G | NA | NA | NA | NA | NA | <0.0001 | NA | — | |
| insG319 | NA | NA | NA | NA | 0.002 | <0.0001 | NA | <0.0001 | — |

NA = not significantly associated

TABLE 7
Associations of ABCA1 SNPs with altered
lipid levels and CAD in REGRESS

| Variant | p-value | Variant | p-value |
|---|---|---|---|
| More coronary events | | Less coronary events | |
| G-191C | 0.001 | C-17G | 0.04 |
| C69T | 0.03 | | |
| Increased family history of | | | |
| CAD | | Less MI prior to study | |
| G-191C | 0.01 | C-17G | 0.02 |
| A-1095G | 0.02 | | |
| Increased progression of | | | |
| atherosclerosis | | Less atherosclerosis | |
| C69T | 0.01 | InsG319 | <0.001 |
| A-1095G (focal | 0.01 | | |
| atherosclerosis) | | | |
| Increased TG | | | |
| C117G | 0.003 | | |

### REFERENCES

1. Murray C J L, Lopez A D. Mortality by cause for eight regions of the world: Global Burden of Disease Study. Lancet. 1997;349:1269-1276.
2. Corti M-C, Barbato G-M, Baggio G. Lipoprotein alterations and atherosclerosis in the elderly. Curr Opin Lipidol. 1997;8:236-241.
3. Manninen V, Tenkanen L, Koskinen P et al. Joint effects of serum triglyceride and LDL cholesterol and HDL cholesterol concentrations on coronary heart disease risk in the Helsinki Heart Study. Implications for treatment. Circulation. 1992;85:37-45.
4. Goldbourt U, Yaari S, Medalie J H. Isolated low HDL cholesterol as a risk factor for coronary heart disease mortality. A 21 year follow-up of 8000 men. Arterioscler Thromb Vasc Biol. 1997; 17:107-113.
5. Campos H, Roederer G O, Lussier-Cacan S et al. Predominance of large LDL and reduced HDL₂ cholesterol in normolipidemic men with coronary artery disease. Arterioscler Thromb Vasc Biol. 1995;15:1043-1048.
6. Schaefer E J, Lamon-Fava S, Ordovas J M et al. Factors associated with low and elevated plasma high density lipoprotein cholesterol and apolipoprotein A-I levels in the Framingham Offspring Study. J Lipid Res. 1994;35:871-882.
7. Fielding C J, Fielding P E. Molecular physiology of reverse cholesterol transport. J Lipid Res. 1995;36:211-228.
8. Hill S A, McQueen M J. Reverse cholesterol transport—A review of the process and its clinical implications. Clin Biochem. 1997;30:517-525.
9. Brooks-Wilson A, Marcil M, Clee S M et al. Mutations in ABC1 in Tangier disease and familial high-density lipoprotein deficiency. Nat Genet. 1999;22:336-345.
10. Marcil M, Brooks-Wilson A, Clee S M et al. Mutations in the ABC1 gene in familial HDL deficiency with defective cholesterol efflux. Lancet. 1999;354:1341-1346.
11. Bodzioch M, Orsó E, Klucken J et al., The gene encoding ATP-binding cassette transporter 1 is mutated in Tangier Disease. Nat Genet. 1999;22:347-351.
12. Rust S, Rosier M, Funke H et al., Tangier Disease is caused by mutations in the gene encoding ATP-binding cassette transporter 1. Nat Genet. 1999;22:352-355.
13. Fredrickson D S, Altrocchi P H, Avioli L V et al., Tangier Disease. Combined clinical staff conference at the National Institutes of Health. Ann Intern Med. 1961;55:1016-1031.
14. Clee S M, Kastelein J J P, van Dam M et al. HDL cholesterol levels and coronary artery disease in heterozygotes for ABC1 mutations are predicted by cholesterol efflux levels and influenced by age. J Clin Invest. 2000;106:1263-1270.
15. Clee S M, Zwinderman A H, Engert J C et al., Common genetic variation in ABC1 is associated with altered lipoprotein levels and a modified risk for coronary artery disease. Circulation. 2001;103:1198-1205.
16. Ehrenborg E, Clee S M, Pimstone S N et al. Ethnic variation and in vivo effects of the −93 t-->g promoter variant in the lipoprotein lipase gene. Arterioscler Thromb Vasc Biol. 1997;17:2672-2678.
17. Pihlajamäki J, Karjalainen L, Karhap\ddot{aa} P et al., G-250A substitution in promoter of hepatic lipase gene is associated with dyslipidemia and insulin resistance in healthy control subjects and in members of families with familial combined hyperlipidemia. Arterioscler Thromb Vasc Biol. 2000;20:1789-1795.
18. Lundahl B, Leren T P, Ose L et al., A functional polymorphism in the promoter region of the microsomal triglyceride transfer protein (MTP-493G/T) influences lipoprotein phenotype in familial hypercholesterolemia. Arterioscler Thromb Vasc Biol. 2000;20:1784-1788.
19. Cavelier L B, Qiu Y, Bielicki J K et al. Regulation and activity of the human ABC1 gene in transgenic mice. J. Biol. Chem. 2001; Papers in Press:Online-March 13.
20. Schork N J, Fallin D, Lanchbury S. Single nucleotide polymorphisms and the future of genetic epidemiology. Clin Genet. 2000;58:250-264.
21. Pullinger C R, Hakamata H, Duchateau P N et al. Analysis of hABC1 Gene 5′ End: Additional peptide sequence, promoter region, and four polymorphisms. Biochem Biophys Res Commun. 2000;271:451-455.
22. Jukema J W, Bruschke A V G, van Boven A J et al. Effects of lipid lowering by pravastatin on the progression and regression of coronary artery disease in symptomatic men with normal to moderately elevated serum cholesterol levels. The Regression Growth Evaluation Statin Study (REGRESS). Circulation. 1995;91:2528-2540.
23. Lee L G, Connell C R, Bloch W. Allelic discrimination by nick-translation PCR with fluorogenic probes. Nuc Ac Res. 1993;21:3761-3766.
24. Holland P M, Abramson R D, Watson R et al. Detection of specific polymerase chain reaction product by utilizing the 5′-->3′ exonuclease activity of Thermus aquaticus DNA polymerase. Proc Natl Acad. Sci. 1991;88:7276-7280.
25. Shen L X, Basilion J P, Stanton V P, Jr. Single-nucleotide polymorphisms can cause different structural folds of mRNA. Proc Natl Acad. Sci. 1999;96:7871-7876.
26. Maquat L E. The power of point mutations. Nat Genet. 2001;27:5-6.
27. Cavalli-Sforza L L. The DNA revolution in population genetics. Trends Genet. 1998;14:60-65.
28. McGraw D W, Forbes S L, Kramer L A et al., Polymorphisms fo the 5′ leader cistron of the human ?₂-adrenergic receptor regulate receptor expression. J Clin Invest. 1998; 102:1927-1932.

1. A method for determining propensity toward developing a cardiovascular disease in a patient at risk of developing said disease comprising determining the presence in an ABCA1 gene of said patient of a polymorphism in the DNA sequence of said gene wherein said polymorphism is present in a non-coding region of said gene.

2. The method of claim 1 wherein said polymorphism is present in the promoter region of said gene.

3. The method of claim 1 wherein said polymorphism is present in an intronic region of said gene.

4. The method of claim 1 wherein said disease is coronary artery disease.

5. The method of claim 1 wherein said disease involves increased triglyceride levels in the plasma of said patient.

6. The method of claim 1 wherein said disease involves decreased high density lipoprotein (HDL-C) levels in the plasma of said patient.

7. The method of claim 1 wherein said disease involves elevated cholesterol levels in the plasma of said patient.

8. The method of claim 1 wherein said disease involves decreased lipid transport in the cells of said patient.

9. The method of claim 1 wherein said polymorphism is a single nucleotide polymorphism.

10. The method of claim 9 wherein said polymorphism is a polymorphism shown in Table 1.

11. A method for identifying a modulator of ABCA1 polynucleotide expression comprising:
(a) contacting a compound with a polynucleotide that encodes ABCA1 polypeptide, which polynucleotide comprises a polymorphism in a non-coding region of said polynucleotide, under conditions promoting said contacting and promoting expression of ABCA1 polypeptide by said polynucleotide;
(b) determining the activity of said polynucleotide in expressing said ABCA1 polypeptide after said contacting wherein a difference in the expression of said polynucleotide relative to when said compound and said polynucleotide are not contacted indicates polynucleotide modulating activity,
thereby identifying a modulator of ABCA1 polynucleotide expression.

12. The method of claim 11 wherein said ABCA1 polynucleotide is present in a cell.

13. The method of claim 11 wherein said difference in expression in step (b) is an increase in expression.

14. The method of claim 11 wherein said polymorphism is present in an intronic region of said polynucleotide.

15. The method of claim 11 wherein said polymorphism occurs in a promoter region of said polynucleotide.

16. The method of claim 11 wherein said polymorphism is a single nucleotide polymorphism (SNP).

17 The method of claim 14 wherein said SNP is a member selected from the SNPs shown in Table 1.

18. The method of claim 11 wherein said polymorphism has the effect of decreasing the activity of said polynucleotide.

19. A method of identifying an agent that modulates plasma lipid levels comprising administering to an animal an effective amount of a compound first identified as an ABCA1 modulator using the method of claim 11.

20. The method of claim 19 wherein said compound has the effect of reducing plasma triglyceride levels.

21. The method of claim 19 wherein said compound has the effect of reducing plasma cholesterol levels.

22. The method of claim 19 wherein said compound has the effect of increasing plasma HDL-C levels.

23. A method of treating a patient for cardiovascular disease comprising administering to a patient afflicted therewith of an effective amount of a compound first identified as an ABCA1 modulator using the method of claim 11 or 19.

24. The method of claim 23 wherein said disease is coronary artery disease.

25. The method of claim 23 wherein said disease is atherosclerosis.

26. A method of protecting a patient against developing cardiovascular disease comprising administering to a patient at risk thereof of an effective amount of a compound first identified as an ABCA1 modulator using the method of claim 11 or 19.

27. The method of claim 26 wherein said disease is coronary artery disease.

28. The method of claim 26 wherein said disease is atherosclerosis.

29. A method for identifying a therapeutic agent for administration to a patient in need thereof, comprising comparing a nucleotide sequence of a non-coding region of an ABCA1 gene of said patient to a database that correlates nucleic acid sequences of ABCA1 genes with the effectiveness of therapeutic agents in beneficially regulating lipid levels in a patient, thereby identifying a therapeutic agent for administration to said patient.

30. The method of claim 29 wherein said database comprises ABCA1 nucleotide sequences comprising the polymorphic sequences disclosed in Table 1.

31. A method for identifying a candidate for enrolment in a program of clinical trials of a potential therapeutic agent, comprising comparing a nucleotide sequence of a non-coding region of an ABCA1 gene of said candidate to a database that correlates nucleic acid sequences of ABCA1 genes with the effectiveness of therapeutic agents in beneficially regulating lipid levels in a patient, thereby identifying a candidate for enrolment in a program of clinical trials.

32. The method of claim 31 wherein said database comprises ABCA1 nucleotide sequences comprising the polymorphic sequences disclosed in Table 1.

33. A method for producing a product comprising identifying an agent according to the process of claim 11 or 19 wherein said product is the data collected with respect to said agent as a result of said process and wherein said data is sufficient to convey the chemical structure and/or properties of said agent.


SEQUENCE LISTING (24 sequences)

SEQ ID NO: 1 - DNA, 22 bp, Homo sapiens
cagcgcttcccgcgcgtcttag

SEQ ID NO: 2 - DNA, 24 bp, Homo sapiens
ccactcactctcgtccgcaattac

SEQ ID NO: 3 - DNA, 38 bp, Homo sapiens
ctgctgagtgactgaactacataaacagaggccgggta

SEQ ID NO: 4 - DNA, 24 bp, Homo sapiens
ccactcactctcgtccgcaattac

SEQ ID NO: 5 - DNA, 22 bp, Homo sapiens
cagcgcttcccgcgcgtcttag

SEQ ID NO: 6 - DNA, 24 bp, Homo sapiens
ccactcactctcgtccgcaattac

SEQ ID NO: 7 - DNA, 20 bp, Homo sapiens
ctggctttctgctgagtgac

SEQ ID NO: 8 - DNA, 19 bp, Homo sapiens
gatcaaagtccccgaaacc

SEQ ID NO: 9 - DNA, 30 bp, Homo sapiens
actcagttgtataacccactgaaaattagt

SEQ ID NO: 10 - DNA, 23 bp, Homo sapiens
ttctatagatgttatcatctggg

SEQ ID NO: 11 - DNA, 30 bp, Homo sapiens
actcagttgtataacccactgaaaatgagt

SEQ ID NO: 12 - DNA, 23 bp, Homo sapiens
ttctatagatgttatcatctggg

SEQ ID NO: 13 - DNA, 20 bp, Homo sapiens
tcatctaaggcacgttgtgg

SEQ ID NO: 14 - DNA, 20 bp, Homo sapiens
cctcaagcctggagtgactt

SEQ ID NO: 15 - DNA, 20 bp, Homo sapiens
atggcaaacagtcctccaag

SEQ ID NO: 16 - DNA, 20 bp, Homo sapiens
accctagcgctgtgtctctg

SEQ ID NO: 17 - DNA, 20 bp, Homo sapiens
atggcaaacagtcctccaag

SEQ ID NO: 18 - DNA, 20 bp, Homo sapiens
accctagcgctgtgtctctg

SEQ ID NO: 19 - DNA, 20 bp, Homo sapiens
tgtgtgtcctcccttccatt

SEQ ID NO: 20 - DNA, 20 bp, Homo sapiens
cttggaggactgtttgccat

SEQ ID NO: 21 - DNA, 38 bp, Homo sapiens
cccctcctgctttatctttcagttaatgaccagccccg

SEQ ID NO: 22 - DNA, 20 bp, Homo sapiens
atccccaactcaaaaccaca

SEQ ID NO: 23 - DNA, 37 bp, Homo sapiens
gccgctgccttccagggctcccgagccacacgctgcg

SEQ ID NO: 24 - DNA, 20 bp, Homo sapiens
atccccaactcaaaaccaca